(19) United States
(12) Patent Application Publication
(10) Pub. No.: US 2009/0118678 A1
(43) Pub. Date: May 7, 2009

(54) INJECTION NEEDLE

(75) Inventor: Yukio KAWASHIMA, Tochigi (JP)

Correspondence Address: LOWE HAUPTMAN HAM & BERNER, LLP, 1700 DIAGONAL ROAD, SUITE 300, ALEXANDRIA, VA, 22314, US

(73) Assignee: TSK LABORATORY JAPAN, Tochigi (JP)

(21) Appl. No.: 10/595,957

(22) PCT Filed: Nov. 18, 2004
(86) PCT No.: PCT/JP2004/017188

§ 371 Date: Feb. 2, 2007

(30) Foreign Application Priority Data
Nov. 21, 2003 (JP) 2003-428312

(51) Int. Cl. A61M 5/32

(52) U.S. Cl. 604/197

(57) ABSTRACT

A needle cover is moveable in the axial direction of a needle body is included. When a cap covering the needle body is detached from a hub, locking portions on a cap are locked at a first locking portion of the cover and the cover moves to the tip end side of the needle body with the cap, so the tip end side of the needle body is covered by the cover. Therefore, the external force in the bending direction is not directly applied to the needle body when or after the cap is detached. Since the needle cover can be moved to the tip end side of the needle body by locking the cap to the needle cover, no spring is needed to move the cover to the tip end side of the needle body, or form the cover into an extendable and retractable complicated shape.

### RELATED APPLICATIONS

The present application is a national phase of PCT/JP04/017188 filed Nov. 18, 2004 which is based on, and claims priority from, Japanese Application Number 2003-428312, filed Nov. 21, 2003, the disclosures of which are hereby incorporated by reference herein in their entirety.

### TECHNICAL FIELD

The present invention relates to an injection needle used in injection of drug solutions, blood collection, dialysis and the like.

### BACKGROUND ART

Generally, when an injection needle is inserted into a patient, the skin of the patient is torn by the needle point and pushed to open by the thickness of the needle tube, and therefore, the patient is give pain in many cases. Thus, by making the outside diameter of the needle tube small, the pain of the patient can be relieved, but the needle tube easily bends as the needle tube becomes thinner, and therefore, when those unskilled in medical activity use injectors, for example, when patients inject drug solutions such as insulin by themselves, useless external force in a bending direction is applied to needles on detaching the caps of the injection needles, thus causing the fear of needle bending and needle breakage.

Thus, as an injection needle to prevent needle bending and needle breakage, the one which has a cylindrical needle cover movable in an axial direction of the needle body attached thereon, with the needle cover biased in a needle point direction by a spring is known (for example, see Patent Document 1). Another injection needle includes an accordion-shaped needle cover extendable and retractable in an axial direction of the needle body, to cover the needle body by extending the needle cover is known (for example, see Patent Document 2).

However, the former has a problem that the number of components becomes large by the spring in use, which makes the manufacturing cost high. The latter has the problem that to extend and retract the needle cover itself, the needle cover has the complicated accordion shape, and work at the time of manufacture is not easy.

Patent Document 1: Japanese Patent Publication 3-139363

Patent Document 2: Japanese Patent Publication 6-16295

### DISCLOSURE OF THE INVENTION

The present invention is made in view of the above described problems, and it is an object of the present invention to provide a new and improved injection needle capable of preventing needle bending and needle breakage and being manufactured easily at low cost.

In order to achieve the above described object, in an injection needle, which includes a fitting member fitted to a parenteral solution discharge port of an injector body, a needle body held by the fitting member, and a cap detachably attached to the fitting member to cover the needle body. The needle body is used by fitting the fitting member with the cap attached thereon to the injector body, and detaching the cap from the fitting member. A cover member movable in an axial direction of the aforesaid needle body exposes a tip end side of the needle body by moving to a base end side of the needle body, and covers the tip end side of the needle body by moving to the tip end side of the needle body. The aforesaid cap has a locking portion that is locked at the cover member to move the cover member to the tip end side of the needle body with the cap.

Thereby, when the cap is detached from the fitting member, the locking portion of the cap is locked at the cover member, and the cover member moves to the tip end side of the needle body with the cap, so that the tip end side of the needle body is covered with the cover member. Therefore, an external force in the bending direction is not directly applied to the needle body when the cap is detached or after it is detached. In addition, it is not necessary to use a spring to move the cover member to the tip end side of the needle body, or to form the cover member into an extendable and retractable complicated shape.

When the cap is detached, the tip end side of the needle body is covered with the cover member. Therefore, an external force in the bending direction is not directly applied to the needle body when the cap is detached or after it is detached, and needle bending and needle breakage can be then reliably prevented. In this case, the cover member can be moved to the tip end side of the needle body by locking of the cap and the cover member. Therefore, it is not necessary to use a spring for moving the cover member to the tip end side of the needle body, or to form the cover member into an extendable and retractable complicated shape, thus making it possible to manufacture the injection needle easily at low cost.

### BRIEF DESCRIPTION OF THE DRAWINGS

FIG. 1 is a perspective view of an injector to which an injection needle of preferred of the present invention is fitted;

FIG. 2 is a sectional side view of a needle cover and a hub;

FIG. 3 is a sectional side view of a cap;

FIG. 4 is perspective views showing fitting steps of the injection needle;

FIG. 5 includes sectional side views of fitting steps of the injection needle;

FIG. 6 includes sectional side views of fitting steps of the injection needle;

FIG. 7 is a sectional side view of a centesis operation into a human body; and

FIG. 8 includes perspective views of fitting steps of the cap.

### DESCRIPTION OF SYMBOLS

1 . . . injector body, 1a . . . parenteral solution discharge port, 2 . . . hub, 2a . . . extended portion, 3 . . . needle body, 4 . . . needle cover, 4a . . . first locking portion, 5 . . . cap, 5c . . . locking portion.

### DETAILED DESCRIPTION OF THE DRAWINGS

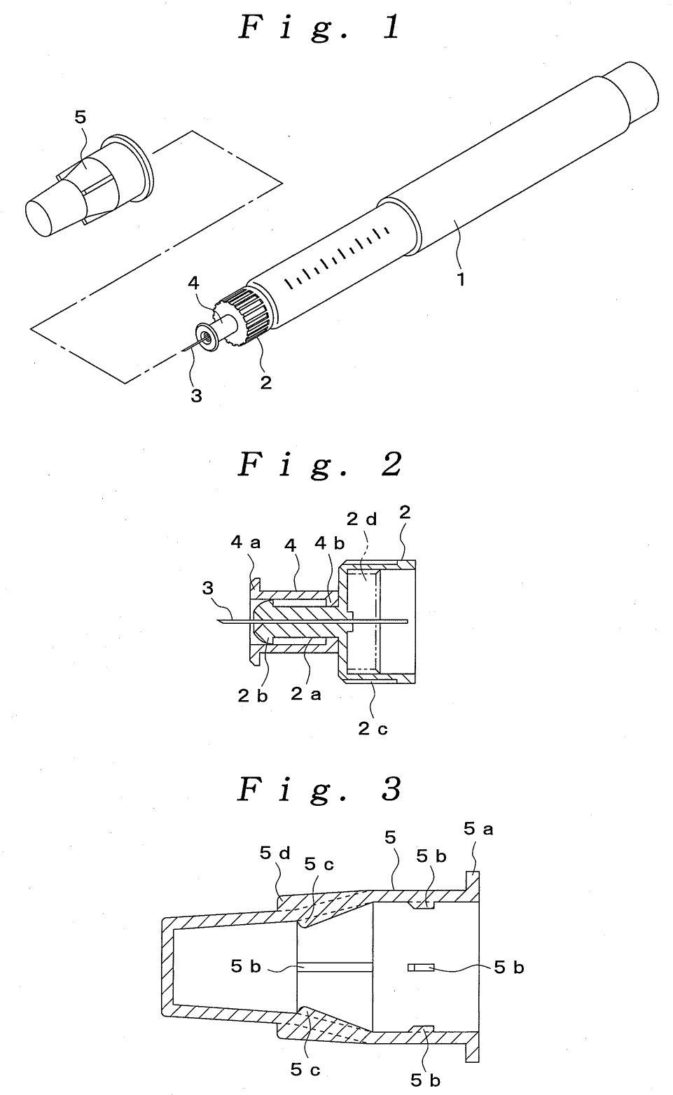

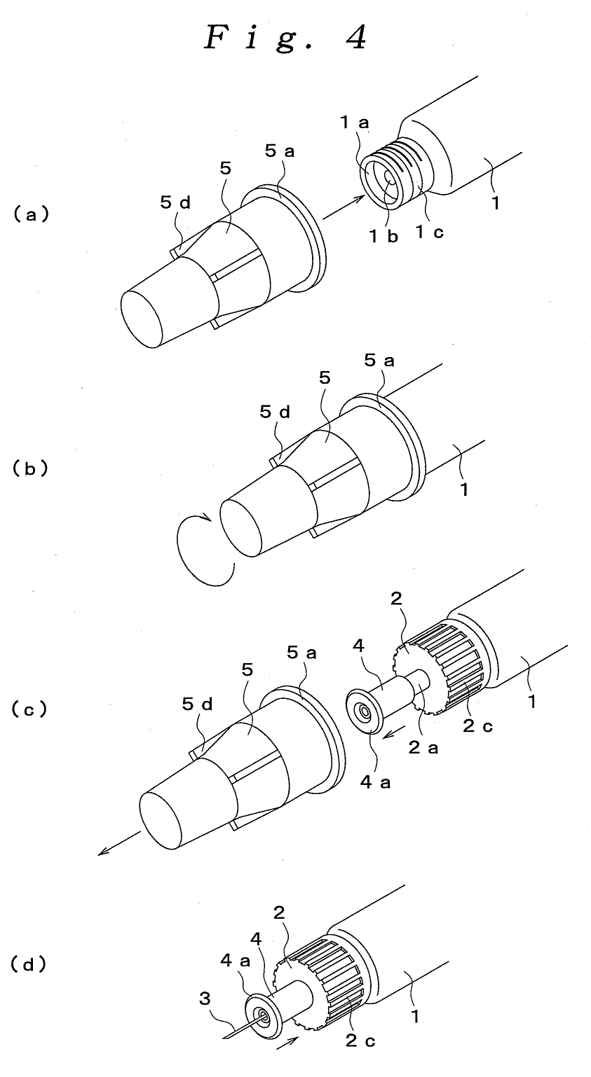

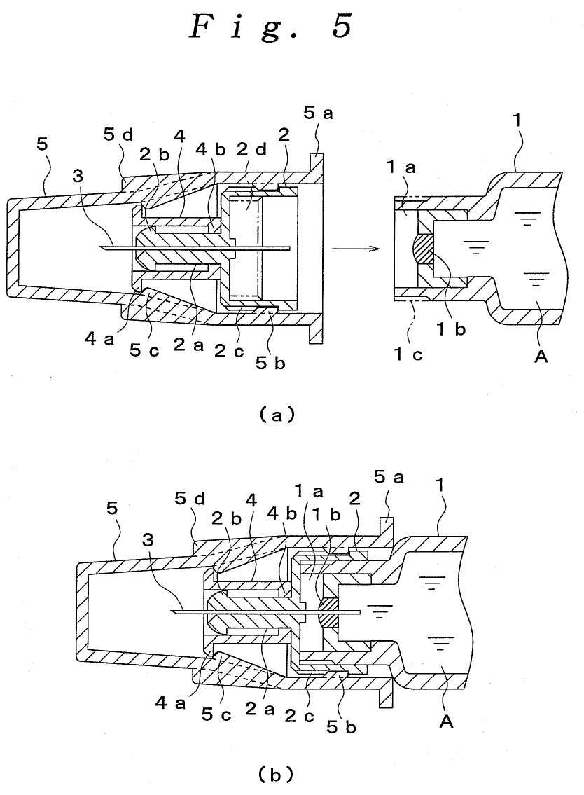

An injection needle shown in FIGS. 1 to 8 includes a hub 2 as a fitting member fitted to an injector body 1, a needle body 3 held by the hub 2, a needle cover 4 as a cover member that is moveable in an axial direction of the needle body 3, and a cap 5 detachably attached to the hub 2 to cover the needle body 3.

The injector body 1 has a parenteral solution discharge port 1a at one end, and a parenteral solution A, such as insulin, that is charged in an inside thereof. The parenteral solution discharge port 1a is closed by a rubber member 1b through which the needle body 3 is capable of penetrating, and a screw portion 1c for fitting the hub 2 is formed on an outer peripheral surface of the parenteral solution discharge port 1a.

The hub 2 includes a cylindrical molded product made of a synthetic resin with one end surface closed, and an extended portion 2a extending in the axial direction at the center of its one end surface. The extended portion 2a extends in the needle point direction to cover the needle body 3 from the one end surface of the hub 2, and the needle body 3 is held by the extended portion 2a. A locking portion 2b which is locked at the needle cover 4 is provided at a tip end side of the extended portion 2a, and the locking portion 2b is formed to expand outward in the diameter direction. A plurality of ribs 2c extending in the axial direction is projectingly provided to be spaced from each other in a circumferential direction on the outer peripheral surface of the hub 2, and a screw portion 2d (simplified in the drawings) capable of being screwed onto the screw portion 1c of the injector body 1 is formed on an inner peripheral surface of the hub 2.

The needle body 3 includes a metal tube with a tip end of the metal tube formed to be sharp, and has its tip end side and base end side projected outward from the extended portion 2a of the hub 2, respectively.

The needle cover 4 includes a cylindrical molded product made of a synthetic resin with both ends of the product opened, and is engaged with the extended portion 2a of the hub 2 movably in the axial direction. A first locking portion 4a which is locked at the cap 5 is at one end side (tip end side of the needle body 3) in the axial direction of the needle cover 4, and the first locking portion 4a is formed to have a flange shape to project outward in the diameter direction. The needle cover 4 has its inside diameter formed to be larger than the outside diameter of the extended portion 2a, and is provided with a second locking portion 4b which is locked at the locking portion 2b of the extended portion 2a at the other end side in its axial direction (base end side of the needle body 3), and the second locking portion 4b is formed to project inward in the diameter direction. Namely, the needle cover 4 moves to the base end side of the needle body 3 along the extended portion 2a to expose the tip end side of the needle body 3, and moves to the tip end side of the needle body 3 to cover the tip end side of the needle body 3. When the needle cover 4 moves to the tip end side of the needle body 3, the second locking portion 4b is locked at the locking portion 2b of the extended portion 2a in the axial direction, so that movement of the needle cover 4 in the needle point direction is restricted.

The cap 5 includes a molded product made of a synthetic resin with one end opened, and a flange portion 5a at its one end opening edge. A plurality of ribs 5b extending in the axial direction is projectingly provided to be spaced in the circumferential direction from each another at one end side of an inner peripheral surface of the cap 5, and each of the ribs 5b is formed to be capable of being locked at the rib 2c of the hub 2 in the circumferential direction. Locking portions 5c capable of being locked at the first locking portion 4a of the needle cover 4 are projectingly provided equidistantly at four spots in total in the circumferential direction at the other end side of the inner peripheral surface of the cap 5. In this case, the space between the locking portions 5copposed to each other in the diameter direction of the cap 5 is formed to be a little smaller than the outside diameter of the first locking portion 4a of the needle cover 4, and by forcefully moving the cap 5 in the axial direction, each of the locking portions 5c is elastically deformed in the diameter direction to ride over the first locking portion 4a of the needle cover 4. A plurality of ribs 5d extending in the axial direction is projectingly provided to be spaced from each other in the circumferential direction on the outer peripheral surface of the cap 5.

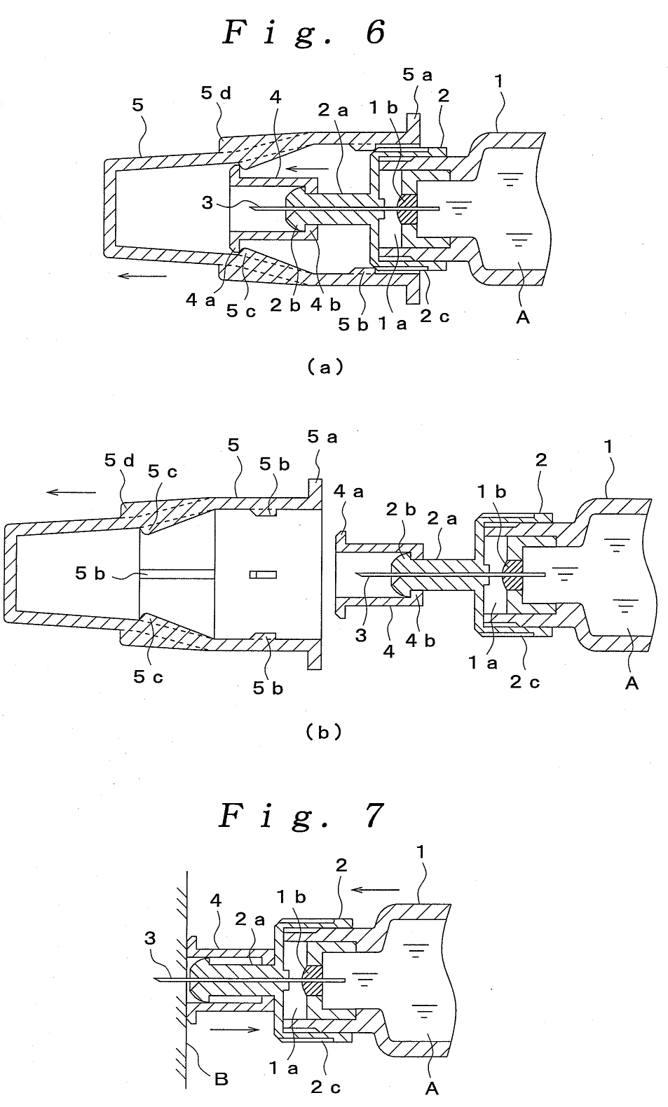

The injection needle having the above construction is accommodated in the cap 5 with the hub 2, the needle body 3 and the needle cover 4 assembled to each other as shown in FIG. 5(a), and the cap 5 is sealed with a lid (not shown) that covers the opening. In this case, each of the first locking portions 5c of the cap 5 is located at the base end side of the needle body 3 with respect to the first locking portion 4a of the needle cover 4.

When the above described injection needle is used, cap 5 is opened by removing the lid (not shown), and the hub 2 is fitted to the parenteral solution discharge port 1a of the injector body 1 with the cap 5 attached as shown in FIG. 5(b). In this case, the screw portion 2d of the hub 2 is screwed onto the screw portion 1c of the injector body 1 by rotating the cap 5 around the axis, and thereby, the hub 2 is fitted to the injector body 1. On this occasion, the rib 5b of the cap 5 is locked at the rib 2c of the hub 2 in the circumferential direction, whereby the cap 5 and the hub 2 are integrally rotated, and each of the ribs 5d on the outer peripheral surface of the cap 5 is a non-slip surface for the finger tips gripping the cap 5.

Next, when the cap 5 is moved in the needle point direction as shown in FIG. 6(a), the needle cover 4 moves in the needle point direction with the cap 5 because each of the locking portions 5c of the cap 5 is locked at the first locking portion 4a of the needle cover 4 from the base end side of the needle body 3, and the tip end side of the needle body 3 is then covered with the needle cover 4. When the force in the needle point direction is further applied to the cap 5, each of the locking portions 5c of the cap 5 rides over the first locking portion 4a of the needle cover 4, and the cap 5 is detached, as shown in FIG. 6(b). Since on this occasion, the tip end side of the needle body 3 is covered with the needle cover 4, the cap 5 does not directly contact the needle body 3 even if the cap 5 is to be contacted to it erroneously when detached, and an external force in the bending direction is not applied to the needle body 3. The needle body 3 is covered with the needle cover 4, and therefore, the finger tips do not directly contact the needle body 3.

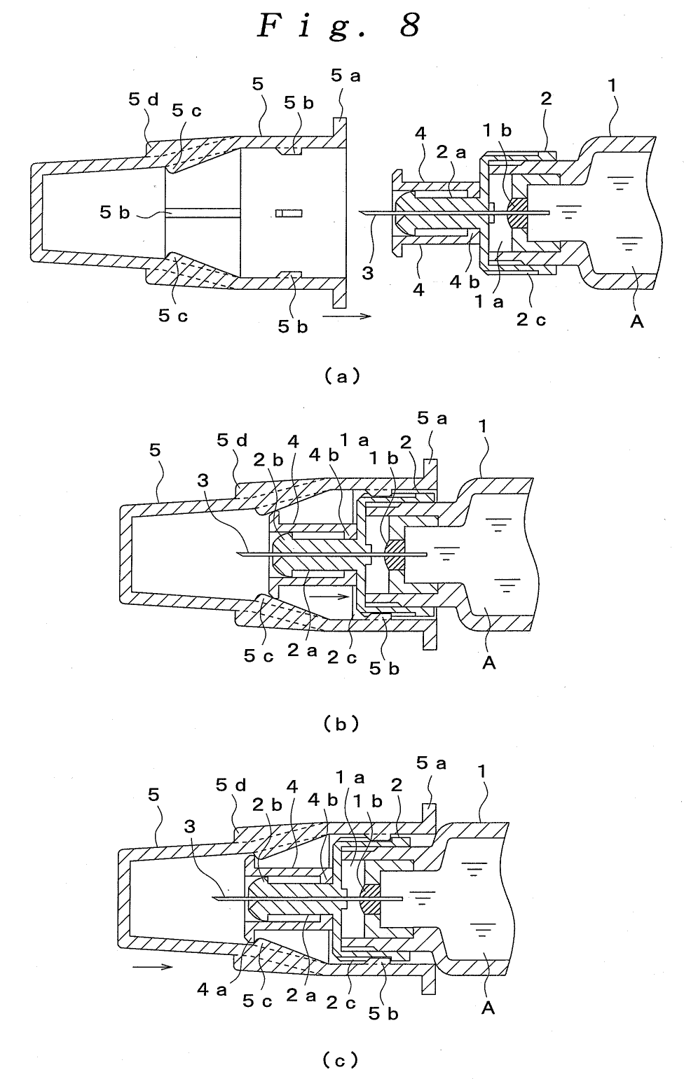

After the cap 5 is detached and with the tip end of the needle body 3 inserted into a human body B of a patient, as shown in FIG. 7, the needle cover 4 moves with respect to the extended portion 2a and is inhibited from moving to the human body B side by abutment on the human body B. Thereby, the drug solution A in the injector body 1 is dischargeable into the human body B from the needle body 3. After completion of the injection, the needle cover 4 is moved in the needle point direction to cover the tip end side of the needle body 3, and thereby, the safety is ensured.

When the cap 5 is to be attached after use, by putting the cap 5 on the needle body 3 side as shown in FIG. 8(a), each of the locking portions 5c of the cap 5 is locked at the first locking portion 4a of the needle cover 4 from the tip end side of the needle body 3, and the needle cover 4 moves in the base end direction of the needle body 3 with the cap 5 to abut one end surface of the hub 2 as shown in FIG. 8(b). When the force in the base end direction of the needle body 3 is further applied to the cap 5, each of the locking portions 5c of the cap 5 is elastically deformed outwardly in the diameter direction of the cap 5 to ride over the first locking portion 4a of the needle cover 4, as shown in FIG. 8(c), and thereby the cap 5 is attached.

When the used injection needle is to be discarded, the cap 5 is rotated in the opposite direction from that at the time of fitting the cap 5 to release the screwed state of the hub 2 and the injector body 1, and thereby, the cap 5 is detached with the hub 2, the needle body 3 and the needle cover 4 accommodated in the cap 5. On this occasion, each of the locking portions 5c of the cap 5 is located at the base end side of the needle body 3 with respect to the first locking portions 4a of the needle cover 4. Therefore, the hub 2, the needle body 3 and the needle cover 4 do not fall off the cap 5 due to locking of the first locking portion 4a and each of the locking portions 5c.

As described above, according to the injection needle of the embodiment, the needle cover 4 is moveable in the axial direction of the needle body 3. When the cap 5 covering the needle body 3 is detached from the hub 2, the locking portions 5c provided at the cap 5 are locked at the first locking portion 4a of the needle cover 4, and the needle cover 4 moves to the tip end side of the needle body 3 with the cap 5, so that the tip end side of the needle body 3 is covered with the needle cover 4. Therefore, an external force in the bending direction is not directly applied to the needle body 3 when the cap 5 is detached or after it is detached, and needle bending and needle breakage can be reliably prevented. Also the needle cover 4 can be moved to the tip end side of the needle body 3 by locking the cap 5 and the needle cover 4. It is not necessary to use a spring for moving the needle cover 4 to the tip end side of the needle body 3, or to form the needle cover 4 into an extendable and retractable complicated shape, and the injection needle can be easily manufactured at low cost.

Further, since the needle cover 4 has a cylindrical shape and engages the extended portion 2a at the hub 2 that is moveable in the axial direction, the needle cover 4 can be reliably moved in the axial direction of the needle body 3 due to the simple structure, and work at the time of manufacture can be performed extremely easily.

Also the locking portion 5c of the cap 5 is formed to ride over the first locking portion 4a of the needle cover 4 in the axial direction due to the elastic deformation, the locking operation and the locking releasing operation of the cap 5 and the needle cover 4 can be made easily, and operability can be enhanced.

In this case, the first locking portion 4a of the needle cover 4 has a flange shape to project in the diameter direction of the needle cover 4, and the locking portions 5c of the cap 5 are projectingly spaced from each other in the circumferential direction at a plurality of spots of the inner peripheral surface of the cap 5. Therefore, the locking portions 5c of the cap 5 can be locked at any location in the circumferential direction of the first locking portion 4a of the needle cover 4, and the cap 5 and the needle cover 4 can be reliably locked.

1. An injection needle comprising:
a fitting member fitted to a parenteral solution discharge port of an injector body;
a needle body held by the fitting member;
a cap detachably attached to the fitting member to cover the needle body, the cap being arranged to fit the fitting member with the cap attached thereon to the injector body and to detach the cap from the fitting, member;
a cover member moveable in an axial direction of said needle for (a) exposing tip end side of the needle body by moving to a base end side of the needle body, and (b) covering the tip end side of the needle body by moving to the tip end side of the needle body;
said cap including a locking portion that is lockable at the cover member to move the cover member to the tip end side of the needle body with the cap.

2. The injection needle according to claim 1, wherein
said fitting member includes an extended portion extending in the axial direction toward the tip end side of the needle body; and
said cover member having a cylindrical shape and engagable with the extended portion moveable in the axial direction.

3. The injection needle according to claim 1, wherein
a locking portion for locking the locking portion of the cap at a predetermined position in the axial direction of said cover member; and
the locking portion of the cap being arranged to ride over the locking portion of the cover member in the axial direction by elastic deformation.

4. The injection needle according to claim 3, wherein
the locking portion of said cover member includes a flange projecting in a radial direction of the cover member; and
the locking portions of said cap being projectingly arranged to be spaced from each other in a circumferential direction at a plurality of spots on an inner peripheral surface of the cap.

5. The injection needle according to claim 1, wherein
a locking portion for locking the locking portion of the cap at a predetermined position in the axial direction of said cover member; and
the locking portion of the cap being arranged to ride over the locking portion of the cover member in the axial direction by elastic deformation.

6. The injection needle according to claim 5, wherein
the locking portion of said cover member includes a flange projecting in a radial direction of the cover member; and
the locking portions of said cap being projectingly arranged to be spaced from each other in a circumferential direction at a plurality of spots on an inner peripheral surface of the cap.
